(19) United States
(12) Patent Application Publication
(10) Pub. No.: US 2026/0076706 A1
(43) Pub. Date: Mar. 19, 2026

(54) SURGICAL TREATMENT APPARATUS

(71) Applicant: FUJIFILM Corporation, Tokyo (JP)

(72) Inventors: Hiroyuki YAEGASHI, Kanagawa (JP); Kohei Higashi, Kanagawa (JP); Keisuke Aoshima, Kanagawa (JP)

(73) Assignee: FUJIFILM Corporation, Tokyo (JP)

(21) Appl. No.: 19/396,345

(22) Filed: Nov. 21, 2025

Related U.S. Application Data
(63) Continuation of application No. PCT/JP2024/019510, filed on May 28, 2024.

(30) Foreign Application Priority Data
May 31, 2023 (JP) 2023-089948

(51) Int. Cl. A61B 17/32; A61B 18/00; A61B 18/08

(52) U.S. Cl. CPC A61B 17/320092; A61B 18/085; A61B 2017/320078; A61B 2017/320082; A61B 2017/320095; A61B 2018/0063

(57) ABSTRACT

A gripping part includes an ultrasound oscillator including a piezoelectric element, a backing member that supports the piezoelectric element, and a stress concentration structure, the piezoelectric element is driven by an impedance matching circuit, and an amount of power supplied to the piezoelectric element is controlled by a processor. The impedance matching circuit drives the piezoelectric element using a first driving frequency and a second driving frequency, and the backing member is fixed to the piezoelectric element in a portion thereof and is not fixed to the piezoelectric element in a different portion thereof.

### CROSS-REFERENCE TO RELATED APPLICATIONS

This application is a Continuation of PCT International Application No. PCT/JP2024/019510 filed on 28 May 2024, which claims priority under 35 U.S.C § 119(a) to Japanese Patent Application No. 2023-089948 filed on 31 May 2023. The above application is hereby expressly incorporated by reference, in its entirety, into the present application.

### BACKGROUND OF THE INVENTION

### 1. Field of the Invention

The present disclosure relates to a surgical treatment apparatus.

### 2. Description of the Related Art

In the medical field, a surgical treatment apparatus that performs various treatments on a subject using a surgical treatment instrument that generates ultrasound vibrations is known. JP2020-534120A (corresponding to WO2019/055870A1) describes a treatment ultrasound device (surgical treatment instrument) in which a transducer (ultrasound oscillator) is provided on a jaw (gripping piece) that grips a biological tissue. The transducer includes a piezoelectric transducer (piezoelectric element), an acoustic matching layer laminated on the piezoelectric transducer, and an air-filled pocket (support member), and the air-filled pocket holds and fixes the entire side surfaces of the piezoelectric transducer.

JP5485967B (corresponding to US2006/0074355A1) describes an ultrasound treatment system comprising an ultrasound transducer (ultrasound oscillator) including a piezoelectric element. In the ultrasound treatment system, the piezoelectric element can be operated at two or more frequencies to cause two or more biological reactions, and as the biological reactions, treatments such as hemostasis and ablation (cauterization) of existing tissue can be performed.

In addition, in a surgical instrument (surgical treatment instrument) described in JP2016-511096A (corresponding to US2014/0276738A1), the gripping piece has a plurality of serrations (stress concentration structures) on a gripping surface facing the biological tissue. As a result, in a case in which the surgical instrument grips the biological tissue, the gripping ability with respect to the biological tissue is improved.

### SUMMARY OF THE INVENTION

In a case in which a sealing treatment of sealing the biological tissue and an incision treatment after the sealing treatment are performed using the surgical treatment apparatus, it is required to smoothly transition from the sealing treatment to the incision treatment using the same surgical treatment instrument. In a case in which the biological tissue is a blood vessel, the sealing treatment also serves as a hemostatic treatment.

Accordingly, the present applicant has conceived providing the stress concentration structure on the gripping piece as in the surgical treatment instrument described in JP2016-511096A, and operating the piezoelectric element at two or more frequencies, as in the ultrasound treatment system described in JP5485967B. Specifically, while gripping biological tissue with the gripping piece of the surgical treatment instrument, the sealing treatment is performed by conducting self-heating of the piezoelectric element to the biological tissue, and then, with the biological tissue remaining gripped by the gripping piece, the frequency is switched to a frequency that contributes to the ultrasound vibration rather than the self-heating of the piezoelectric element, thereby promoting the ultrasound vibration by the piezoelectric element. As a result, the stress concentration structure that concentrates the stress on the biological tissue vibrates, and thus it is assumed that the biological tissue after the sealing is incised using the vibration.

In a case in which it is desired to perform a series of operations in which the frequency is switched from the sealing treatment mainly causing the piezoelectric element to generate heat to the incision treatment by promoting the ultrasound vibration of the piezoelectric element, a problem may arise in that heat generation of the piezoelectric element in the sealing treatment hinders the vibration of the piezoelectric element during the incision treatment. That is, in the course of performing the sealing treatment, moisture contained in the biological tissue enters the interior of the gripping part and solidifies and adheres in a gap between the self-heated piezoelectric element and the gripping piece, thereby hindering the vibration of the piezoelectric element during the incision treatment. This may result in problems of poor incision by the surgical treatment instrument.

In the surgical treatment instrument described in JP2020-534120A and JP2016-511096A, it is not considered that the incision treatment is performed by switching the frequency, and in the ultrasound treatment system described in JP5485967B, it is not considered that the biological tissue adheres to the piezoelectric element due to the sealing treatment.

An object of the present disclosure is to provide a surgical treatment apparatus that can prevent the moisture contained in the biological tissue that has entered an inner portion of the gripping part from adhering to the piezoelectric element and hindering the ultrasound vibration of the piezoelectric element.

An aspect of the present disclosure relates to a surgical treatment apparatus comprising: a gripping part; an ultrasound oscillator; a support member; an impedance matching circuit; a processor; and a stress concentration structure, in which the impedance matching circuit drives a piezoelectric element using a first driving frequency and a second driving frequency that are different from each other, and the support member is in a state fixed to the piezoelectric element in a portion among portions that support the piezoelectric element, and is in a state not fixed to the piezoelectric element in a portion different from the portion. The gripping part grips a biological tissue. The ultrasound oscillator is provided at the gripping part and includes the piezoelectric element. The support member is provided at the gripping part and supports the piezoelectric element. The impedance matching circuit is for driving the piezoelectric element. The processor controls an amount of power supplied to the piezoelectric element. The stress concentration structure is provided at the gripping part and protrudes from a gripping surface facing the biological tissue.

It is preferable that a non-fixed portion of the support member that is not fixed to the piezoelectric element be disposed at a position at which the biological tissue does not enter in a case in which the gripping part grips the biological tissue.

It is preferable that the non-fixed portion support the piezoelectric element on an inner side of an end surface of the piezoelectric element in a width direction intersecting a vibration direction of the piezoelectric element.

It is preferable that fixed portions of the support member that are fixed to the piezoelectric element be fixed to the piezoelectric element at positions equidistant from a center line of the piezoelectric element in the width direction.

It is preferable that the gripping part include a pair of gripping pieces that grip the biological tissue, and the ultrasound oscillator and the stress concentration structure be provided on at least one of the gripping pieces.

It is preferable that the gripping part include a pair of gripping pieces that grip the biological tissue, and the stress concentration structure be provided on at least one of the gripping pieces.

It is preferable that the support member be disposed parallel to a longitudinal direction intersecting the vibration direction of the piezoelectric element and the width direction.

It is preferable that the fixed portions be located at a center of the piezoelectric element in the longitudinal direction. It is preferable that the non-fixed portion have a hydrophobic surface. It is preferable that the biological tissue be a blood vessel.

According to the aspect of the present disclosure, it is possible to prevent the moisture contained in the biological tissue that has entered the inner portion of the gripping part from hindering the ultrasound vibration of the piezoelectric element.

### BRIEF DESCRIPTION OF THE DRAWINGS

FIG. 1 is a diagram illustrating a configuration of a surgical treatment apparatus and an endoscope system.

FIG. 2 is a side view illustrating a use state in which a surgical treatment instrument is inserted into a forceps channel of an endoscope.

FIGS. 3A and 3B are side views of the vicinity of a gripping part of the surgical treatment instrument, FIG. 3A illustrating a state in which the gripping part is closed and FIG. 3B illustrating a state in which the gripping part is opened.

FIG. 4 is a perspective view of the surgical treatment instrument in a state in which the gripping part is closed.

FIG. 5 is a perspective view of the surgical treatment instrument in a state in which the gripping part is opened.

FIG. 6 is an exploded perspective view of an ultrasound oscillator unit and a gripping piece.

FIG. 7 is a main part cross-sectional view of the gripping part taken along line VII-VII of FIG. 3A.

FIG. 8 is a main part cross-sectional view of the gripping part taken along line VIII-VIII of FIG. 3A.

FIG. 9 is a perspective view of the vicinity of an ultrasound oscillator and a stress concentration structure.

FIG. 10 is a main part cross-sectional view of the vicinity of the ultrasound oscillator and the stress concentration structure.

FIG. 11 is a plan view of a backing member.

FIG. 12 is a plan view of the ultrasound oscillator unit in which the ultrasound oscillator is attached to the backing member.

FIG. 13 is a block diagram illustrating an outline of a drive device.

FIG. 14 is a perspective view of a piezoelectric element included in the ultrasound oscillator.

FIG. 15 is a main part cross-sectional view illustrating a state before a structure is gripped using the surgical treatment apparatus.

FIG. 16 is a main part cross-sectional view illustrating a state in which the gripping of the structure is started using the surgical treatment apparatus.

FIG. 17 is a main part cross-sectional view illustrating a state in which the structure is sealed using the surgical treatment apparatus.

FIG. 18 is a flowchart illustrating an operation of the surgical treatment apparatus.

### DESCRIPTION OF THE PREFERRED EMBODIMENTS

### Schematic Configuration of Endoscope System

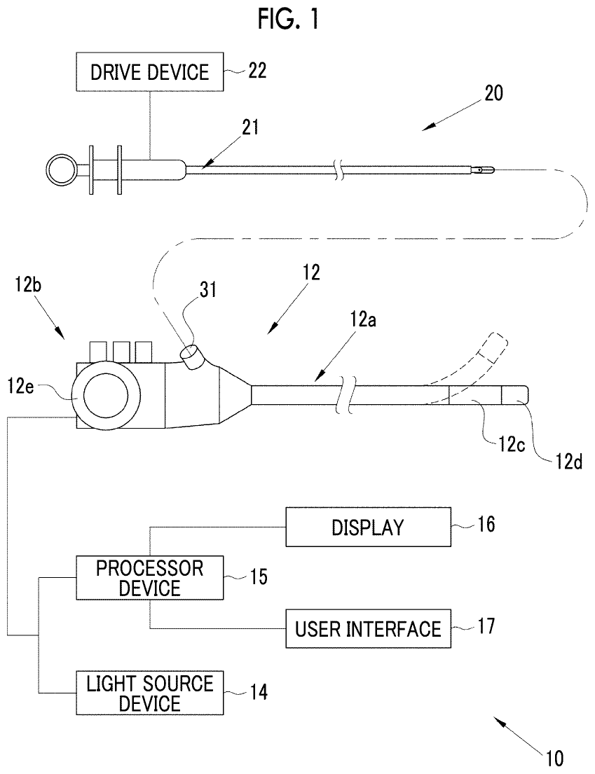

As illustrated in FIG. 1, a surgical treatment apparatus 20 according to an embodiment of the present disclosure is used in combination with an endoscope system 10. The endoscope system 10 comprises an endoscope 12, a light source device 14, a processor device 15, a display 16, and a user interface (UI) 17. The endoscope 12 images an observation target. The light source device 14 emits illumination light for irradiating the observation target. The processor device 15 performs system control of the endoscope system 10. The display 16 is a display unit that displays an observation image and the like based on an endoscopic image. The UI 17 is an input device that performs setting input and the like to the processor device 15 or the like, and is a mouse, a touchpad, and/or a keyboard.

The endoscope 12 is optically connected to the light source device 14 and is electrically connected to the processor device 15. The endoscope 12 has an insertion part 12a to be inserted into a subject, an operating part 12b provided at a base end part of the insertion part 12a, a bendable part 12c provided on a distal end side of the insertion part 12a, and a distal end part 12d. The bendable part 12c is bent by operating an angle knob 12e of the operating part 12b. As a result, the distal end part 12d faces a desired direction.

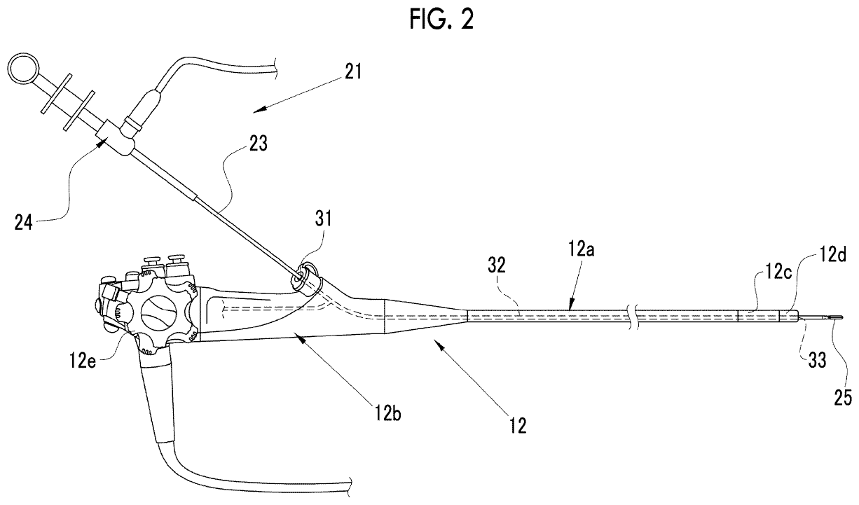

The operating part 12b is provided with a forceps port 31 (see FIG. 2) in addition to the angle knob 12e. The forceps port 31 is an inlet into which an endoscopic treatment instrument such as a surgical treatment instrument 21 is inserted. The endoscopic treatment instrument is used in a state inserted into the forceps port 31. The endoscope 12 may be a flexible endoscope in which the insertion part 12a is flexible (flexibility), or may be a rigid endoscope in which the insertion part 12a is rigid (rigidity).

An observation window or an illumination window is provided on a distal end surface of the distal end part 12d. An image sensor (not illustrated) or the like is disposed behind the observation window, and an optical fiber cable (not illustrated) is disposed behind the illumination window. A signal line of the image sensor and the optical fiber cable are connected to each of the processor device 15 and the light source device 14.

The processor device 15 is electrically connected to the display 16 and the UI 17. The processor device 15 performs image processing and the like on the endoscopic image captured by the image sensor, and displays the processed endoscopic image on the display 16.

### Schematic Configuration of Surgical Treatment Apparatus

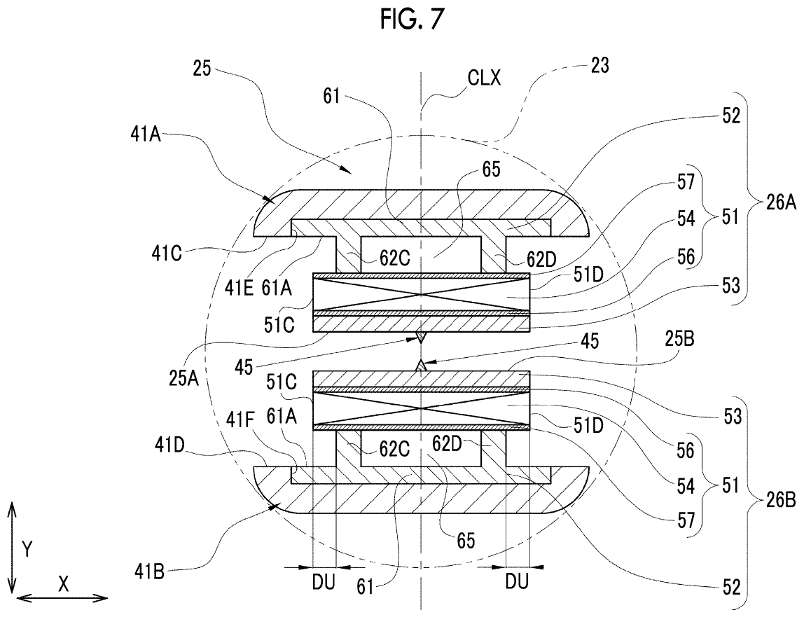

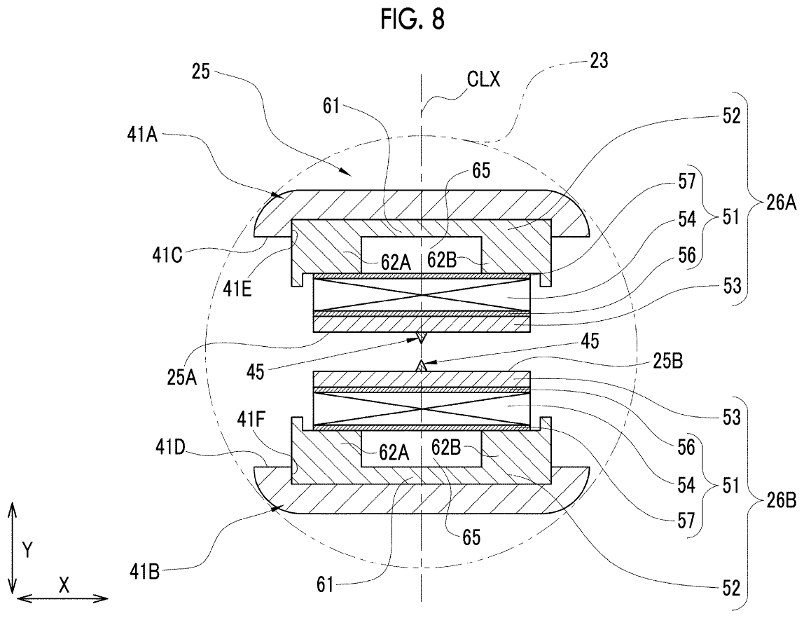

The surgical treatment apparatus 20 comprises a surgical treatment instrument 21, which is an endoscopic treatment instrument inserted into the subject through the endoscope 12, and a drive device 22 that supplies power. The surgical treatment instrument 21 has a flexible sheath 23, an operating part 24, a gripping part 25, and ultrasound oscillator units 26A and 26B (see FIGS. 7 and 8), and is electrically connected to the drive device 22. The flexible sheath 23 is a tubular sheath formed of a flexible material such as a soft resin, and is inserted into a forceps channel 32 of the endoscope 12.

The surgical treatment apparatus 20 is used for, for example, laparoscopic surgery and is used for a treatment such as sealing and incision. The gripping part 25 is located at a distal end of the surgical treatment instrument 21, and grips a biological tissue. Specifically, the biological tissue to be gripped is a soft tissue such as a blood vessel. In the operating part 24, a user operation of adjusting an opening and closing of the gripping part 25 or a gripping strength of the gripping part 25 in a state in which the gripping part 25 is closed, that is, a gripping state is received.

As illustrated in FIG. 2, the forceps channel 32 for inserting the surgical treatment instrument 21 is disposed in the insertion part 12a of the endoscope 12. The forceps channel 32 has one end connected to a forceps outlet 33 and the other end connected to the forceps port 31 provided in the operating part 12b. The gripping part 25 of the surgical treatment instrument 21, which is inserted into the forceps port 31, protrudes from the forceps outlet 33 of the distal end part 12d. The forceps channel 32 is also used as a path for feeding a washing solution such as water from the forceps outlet 33 and a path for suctioning a body fluid such as blood and bodily waste products.

### Configuration of Gripping Part

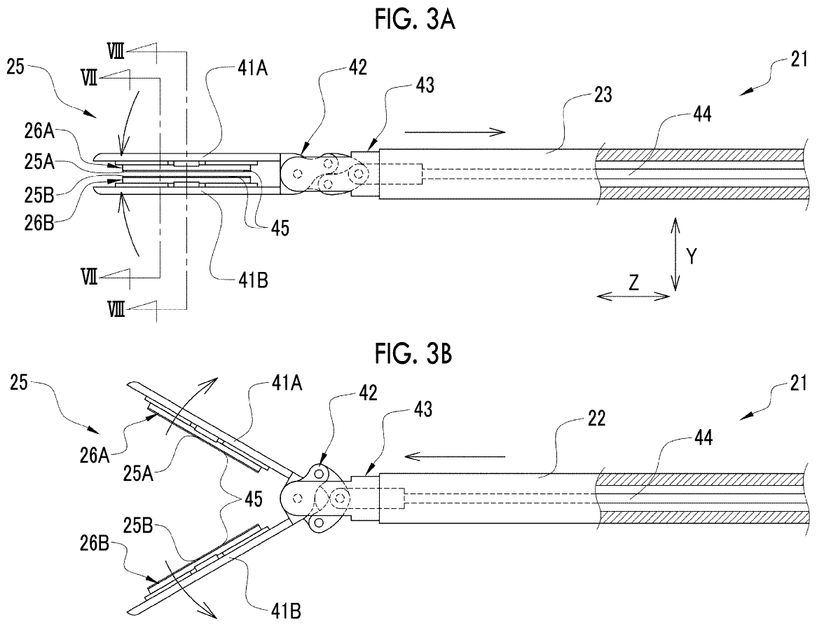

As illustrated in FIGS. 3A and 3B, the gripping part 25 includes a pair of gripping pieces 41A and 41B, an opening/closing mechanism 42, and a support member 43 that supports these. The pair of gripping pieces 41A and 41B are openable and closable in an up-down direction. In addition, the support member 43 is formed in a substantially cylindrical shape, and is fixed to a distal end of the flexible sheath 23.

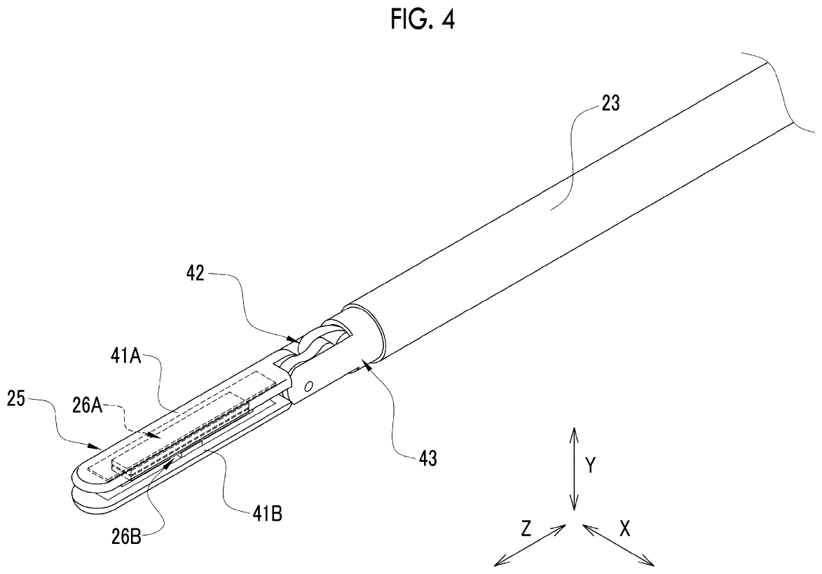

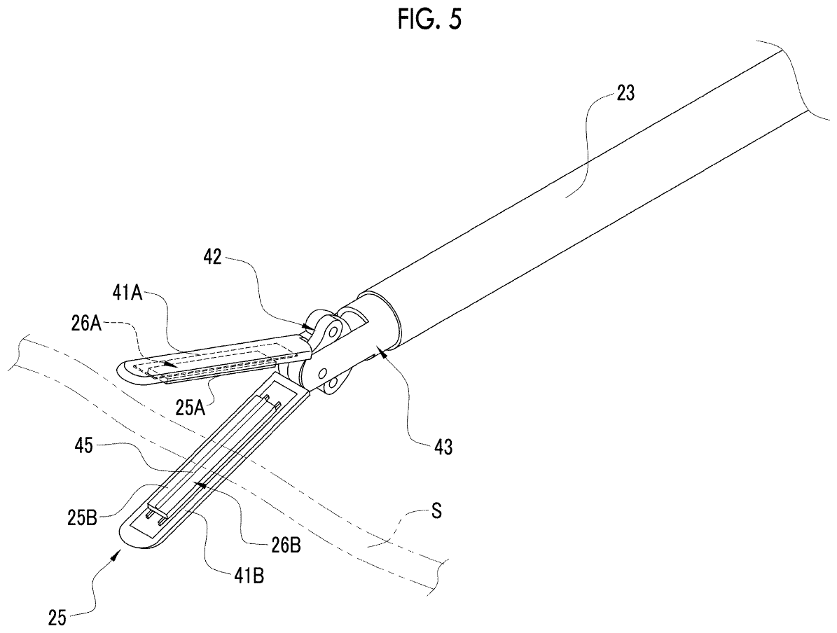

The opening/closing mechanism 42 is connected to an operating wire 44 constituting the operating part 24. The operating wire 44 moves linearly in accordance with the user operation using the operating part 24. The opening/closing mechanism 42 converts the linear movement transmitted from the operating wire 44 into a rotational movement to perform the opening/closing operation of the pair of gripping pieces 41A and 41B. For example, the pair of gripping pieces 41A and 41B is in a closed state in a case in which the operating wire 44 retreats to the operating part 24 side with respect to the flexible sheath 23 and is in an opened state in a case in which the operating wire 44 advances toward the distal end side. As a result, the form of the gripping part 25 can be switched between a state in which the pair of gripping pieces 41A and 41B are closed and the gripping part 25 is closed as illustrated in FIGS. 3A and 4, and a state in which the pair of gripping pieces 41A and 41B are opened and the gripping part 25 is opened as illustrated in FIG. 3B and FIG. 5.

A state in which the gripping part 25 is closed is a state in which the entire gripping part 25 is located on an inner side of an outer peripheral surface of the flexible sheath 23 (a state in which the entire gripping part 25 is located on an inner side of a two-dot chain line circle illustrated in FIGS. 7 and 8). As a result, the gripping part 25 in the closed state can be inserted into the forceps channel 32 of the endoscope 12 in the same manner as the flexible sheath 23. On the other hand, a state in which the gripping part 25 is opened is a state in which at least a portion of the gripping part 25 is located on an outer side of the outer peripheral surface of the flexible sheath 23 (a state in which the at least a portion of the gripping part 25 is located on an outer side of the two-dot chain line circle illustrated in FIGS. 7 and 8).

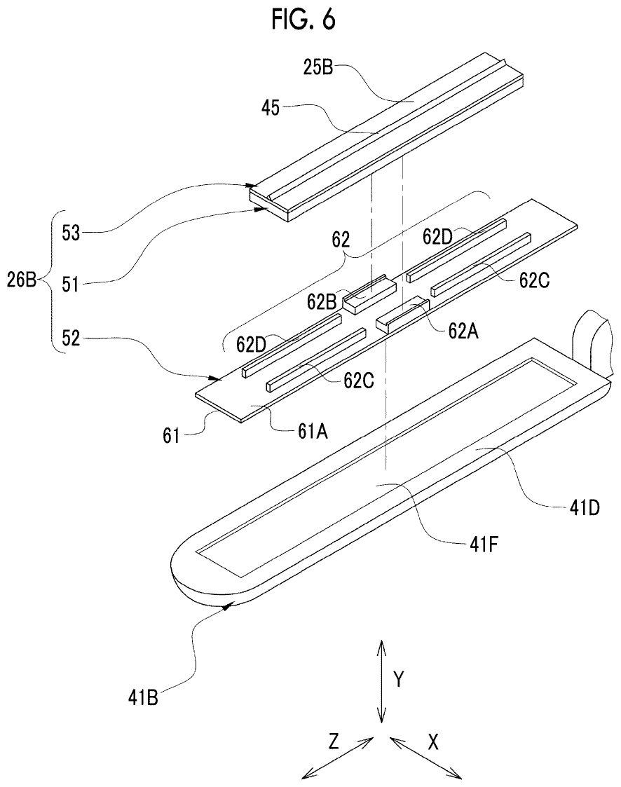

The gripping pieces 41A and 41B are formed in an elongated plate shape, that is, a plate shape in which a dimension in an insertion direction Z is longer than a dimension in a width direction X (see FIGS. 4 and 6), and are formed to have the same outer shape as each other. The shapes of the gripping pieces 41A and 41B are not limited to the above-described example, and may be different from each other. For example, in a case in which the shapes of the ultrasound oscillator units 26A and 26B are different from each other, the shapes of the gripping pieces 41A and 41B that hold the ultrasound oscillator units 26A and 26B may also be different from each other so as to appropriately conform to the shapes of the ultrasound oscillator units 26A and 26B.

The distal end parts of the gripping pieces 41A and 41B in the insertion direction Z have a curved surface shape, and are easily inserted into the forceps channel 32. The insertion direction Z refers to a direction parallel to the insertion direction of the surgical treatment instrument 21 in a state in which the gripping part 25 is closed. In addition, a gripping direction Y, which will be described later, is a direction in which the gripping pieces 41A and 41B grip the biological tissue such as the blood vessel in a state in which the gripping part 25 is closed, and is a direction orthogonal to the insertion direction Z. Further, the width direction X is a direction orthogonal to the insertion direction Z and the gripping direction Y.

A stress concentration structure 45 (see FIGS. 7 and 8) is provided on both of the pair of gripping pieces 41A and 41B. The configuration is not limited to the above, and stress concentration structure 45 may be provided on at least one of the gripping piece 41A or the gripping piece 41B. Details of the stress concentration structure 45 will be described later.

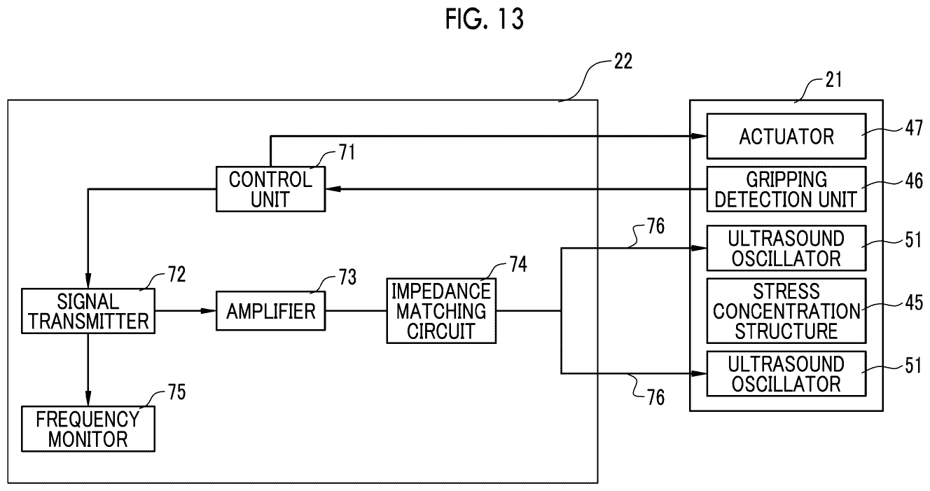

In addition, the surgical treatment instrument 21 comprises a gripping detection unit 46 and an actuator 47 (see FIG. 13). The gripping detection unit 46 and the actuator 47 are connected to a control unit 71 of the drive device 22 via a signal line (not illustrated) or the like. The gripping detection unit 46 is a sensor for detecting whether or not the gripping by the gripping part 25 is present and detects the gripping strength (magnitude of the gripping force). The gripping detection unit 46 is, for example, a pressure sensor that detects a pressure received by gripping a structure S (see FIG. 5), which is the biological tissue such as the blood vessel, by the pair of gripping pieces 41A and 41B, and is provided on the gripping part 25, the opening/closing mechanism 42, or the like. The structure S that is the biological tissue is the blood vessel, and particularly, a blood vessel having an outer diameter of equal to or greater than 3 mm and equal to or less than 5 mm and a thickness of equal to or greater than 0.5 mm and equal to or less than 0.8 mm is assumed.

The actuator 47 is an actuator for holding a state in which the structure S is pressurized and gripped by the gripping part 25, and is, for example, a piezoelectric actuator that operates the pair of gripping pieces 41A and 41B by energizing the piezoelectric element to perform the pressurized gripping by the gripping part 25. The actuator 47 is provided on the gripping part 25, the opening/closing mechanism 42, or the like.

### Configuration of Ultrasound Oscillator Unit

The ultrasound oscillator units 26A and 26B are provided on the gripping part 25 and constitute a portion of the gripping part 25 as described later. Specifically, the ultrasound oscillator unit 26A provided on an inner surface of the gripping piece 41A and the ultrasound oscillator unit 26B provided on an inner surface of the gripping piece 41B are provided at positions facing each other.

As illustrated in FIG. 6, the ultrasound oscillator unit 26B is configured by laminating an ultrasound oscillator 51, a backing member 52, and an acoustic matching layer 53. The backing member 52 corresponds to a support member in the claims. The ultrasound oscillator unit 26A has the same configuration as the ultrasound oscillator unit 26B, and the same components and/or members will be denoted by the same reference numerals and will not be described.

As illustrated in FIGS. 7 and 8, the ultrasound oscillator 51 includes a piezoelectric element 54 and electrode layers 56 and 57. The piezoelectric element 54 is formed in a rectangular plate shape. The electrode layers 56 and 57 are formed in a thinner plate shape than the piezoelectric element 54 and are laminated on both surfaces of the piezoelectric element 54. As an example, a piezoelectric element polarized in a thickness direction can be used as the piezoelectric element 54.

The gripping piece 41B has a recess 41F that is recessed by one step relative to the periphery on an inner surface 41D facing the gripping piece 41A. The ultrasound oscillator 51 is attached to the gripping piece 41B via the backing member 52. The gripping piece 41A also has a recess 41E that is recessed by one step on an inner surface 41C facing the gripping piece 41B and has the ultrasound oscillator 51 attached thereto via the backing member 52, similarly to the gripping piece 41B.

The backing member 52 includes a base part 61. The base part 61 is formed in an elongated rectangular plate shape having a slightly smaller outer shape than the gripping pieces 41A and 41B. The recess 41F of the gripping piece 41B is recessed in a rectangle in accordance with the outer shape of the base part 61. The backing member 52 is fixed to the gripping piece 41B by, for example, fitting the base part 61 into the recess 41F and bonding the base part 61 to the recess 41F with an adhesive. The method of fixing the backing member 52 to the gripping piece 41B is not limited to this method, and the backing member 52 may be fixed to the gripping piece 41B by, for example, screwing or the like. Details of the backing member 52 will be described later.

The backing member 52 fixed to the gripping piece 41B is fixed such that an inner surface 61A (surface on a side facing the gripping piece 41A) of the base part 61 is flush with the inner surface 41D of the gripping piece 41B. As a result, the backing member 52 is disposed parallel to the insertion direction Z that is a longitudinal direction of the ultrasound oscillator 51. The support part 62 is provided to protrude from the inner surface 61A of the base part 61. The base part 61 of the backing member 52 is fixed to the recess 41E of the gripping piece 41A in the same manner as the gripping piece 41B. The backing member 52 fixed to the gripping piece 41A is fixed such that the inner surface 61A (surface on the side facing the gripping piece 41B) of the base part 61 is flush with the inner surface 41D of the gripping piece 41A.

The acoustic matching layer 53 of the ultrasound oscillator unit 26B constitutes a gripping surface 25B of the gripping part 25. Specifically, the ultrasound oscillator 51 is located on an inner side of the gripping piece 41B in the gripping direction Y, and the acoustic matching layer 53 laminated on the ultrasound oscillator 51 is located on an inner side of the ultrasound oscillator 51 in the gripping direction Y. That is, in a case in which the gripping part 25 grips the biological tissue such as the blood vessel, an inner surface of the acoustic matching layer 53 facing the biological tissue is the gripping surface 25B.

The acoustic matching layer 53 of the ultrasound oscillator unit 26A constitutes a gripping surface 25A of the gripping part 25 in the same manner as the ultrasound oscillator unit 26B. That is, in a case in which the gripping part 25 grips the biological tissue such as the blood vessel in the acoustic matching layer 53, an inner surface facing the biological tissue is the gripping surface 25A.

As the piezoelectric element 54, for example, lead zirconate titanate (PZT) is used. The piezoelectric element 54 is not limited thereto, and a piezoelectric element having a mechanical quality factor Qm of equal to or greater than 500 is preferably used. The mechanical quality factor Qm is a factor representing the elasticity loss caused by the vibration and is represented by the reciprocal of a mechanical loss factor. In a case in which the piezoelectric element 54 elastically vibrates, the loss occurs internally and is converted into heat.

In addition, a Curie temperature of the piezoelectric element 54 is preferably equal to or greater than 1.5 times a target reach temperature of the surface of the piezoelectric element 54 during power supply, and more preferably equal to or greater than 2 times. In a case in which this condition of the Curie temperature is satisfied, for example, lithium niobate (LiNbO₃) or barium titanate (BaTiO₃) may be used as the piezoelectric element 54. Since proteins may be carbonized at temperatures exceeding 200° C., it is preferable that the target reach temperature of the surface of the piezoelectric element 54 be set to a maximum of 250° C.

The acoustic matching layer 53 is provided in order to achieve acoustic impedance matching between a human body of a patient and the ultrasound oscillator 51. The acoustic matching layer 53 can increase the transmittance of ultrasonic waves. As a material for the acoustic matching layer 53, various organic materials of which the acoustic impedance has a value closer to a value of the human body as compared to the piezoelectric element 54 can be used. Specific examples of the material include an epoxy resin, silicon rubber, polyimide, and polyethylene. In addition, the acoustic matching layer 53 is formed of a plurality of layers, and the material and the number of formed layers are selected as appropriate in accordance with a required acoustic impedance value.

In addition, since a difference in thermal expansion factor between the piezoelectric element 54 and the acoustic matching layer 53 causes internal stress between the ultrasound oscillator 51 and the acoustic matching layer 53 during temperature increase, which causes variation in the temperature dependence of a thickness resonance frequency and a length resonance frequency of the piezoelectric element 54 described later, the material, dimensions, and the like of the acoustic matching layer 53 are selected in consideration of the thermal expansion factor of the piezoelectric element 54.

The electrode layers 56 and 57 are connected to the drive device 22 via a signal cable 76 (see FIG. 13). The signal cable 76 is provided in the flexible sheath 23. For example, the signal cable 76 is wired along an inner peripheral surface or the outer peripheral surface of the flexible sheath 23. The configuration is not limited to this, and a portion of the flexible sheath 23 may be formed of a conductive material, instead of the signal cable 76. In a case in which the surgical treatment instrument 21 is connected to the drive device 22, the electrode layers 56 and 57 are electrically connected to the drive device 22 via the signal cable 76. One of the electrode layer 56 or the electrode layer 57 is connected to a ground via the signal cable or the like, and power of an alternating-current voltage signal, which will be described later, is supplied from the drive device 22 to the other of the electrode layer 56 or the electrode layer 57.

### Configuration of Stress Concentration Structure

The stress concentration structure 45 is a protrusion that protrudes from the gripping surfaces 25A and 25B. As described above, the gripping surfaces 25A and 25B are formed of the acoustic matching layer 53. Therefore, the stress concentration structure 45 is fixed to the surface of the acoustic matching layer 53.

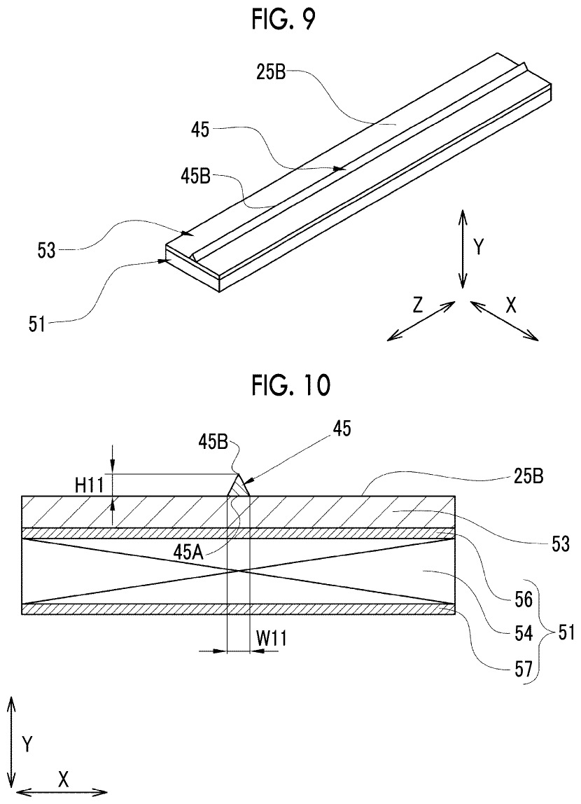

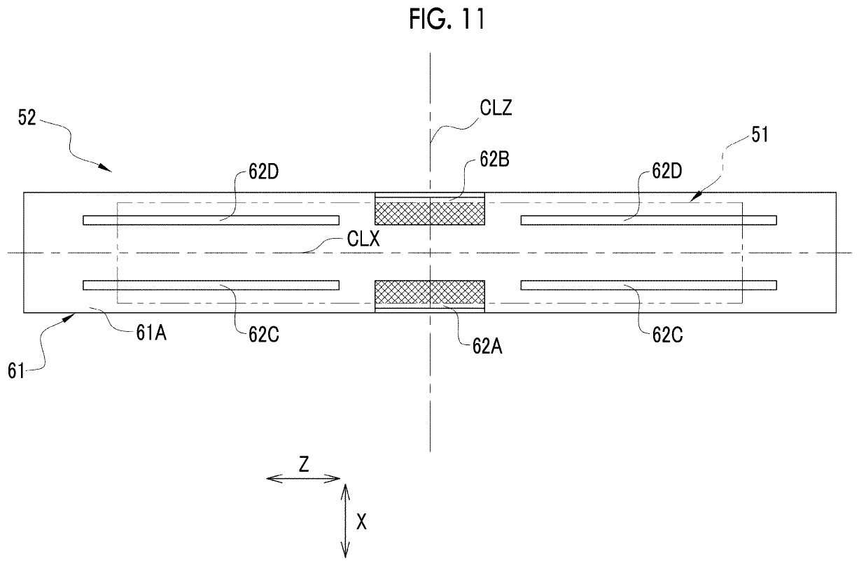

As illustrated in FIG. 9, the stress concentration structure 45 is an elongated protrusion having a triangular prism-like outer shape, the axial direction of which is parallel to the longitudinal direction of the gripping pieces 41A and 41B, that is, the insertion direction Z. Specifically, the stress concentration structure 45 is disposed such that one side surface 45A (see FIG. 10) of three side surfaces constituting the triangular prism is in contact with the acoustic matching layer 53 located on an outer side in the gripping direction Y, and a ridgeline 45B opposite to the side surface in contact with the acoustic matching layer 53 faces an inner side in the gripping direction Y. In the stress concentration structure 45, stress is concentrated at the position of the ridgeline 45B facing the inner side in the gripping direction Y, and the structure S can be reliably gripped.

It is preferable that the stress concentration structure 45 have a shape that does not incise the structure S during pressurized gripping, specifically, a shape in which an apex angle including the ridgeline 45B in which stress is concentrated is not excessively acute and a height dimension from the gripping surfaces 25A and 25B is not excessively high. The stress concentration structure 45 is not limited to the triangular prism shape, but need only have any shape in which stress is concentrated on the structure, and may have a quadrangular prism shape. In addition, in a case in which the stress concentration structure 45 has the quadrangular prism shape, it is preferable that an inner piece facing the biological tissue have a smaller trapezoidal cross section than an outer piece in contact with the acoustic matching layer 53. Alternatively, the stress concentration structure 45 may be formed in a serrated shape on a distal end side (inner side) in the protrusion direction from the gripping surfaces 25A and 25B, that is, the gripping direction Y.

As a method of fixing the stress concentration structure 45 to the acoustic matching layer 53, for example, the stress concentration structure 45 is adhered by applying the adhesive between the acoustic matching layer 53 and the stress concentration structure 45. Alternatively, a groove may be formed on the surface of the acoustic matching layer 53, and the stress concentration structure 45 may be fitted into the groove. Alternatively, the stress concentration structure 45 may be fixed to the acoustic matching layer 53 by both the adhesion and the fitting into the groove. The configuration is not limited to this, and for example, the acoustic matching layer 53 and the stress concentration structure 45 may be integrally formed by metal or the like. In addition, a configuration may be adopted in which a member including the stress concentration structure 45 located at the center of the gripping surfaces 25A and 25B, and ultrasound oscillators respectively adjacent to the left and right of the stress concentration structure 45 and sandwiching the member including the stress concentration structure 45 are provided.

The stress concentration structure 45 has a Brinell hardness of equal to or greater than 40 HBW and is made of, for example, metal or ceramics. As a result, stress is more easily transmitted to the biological tissue gripped by the gripping part 25, and the treatment of incising the biological tissue can be performed more efficiently. Further, the stress concentration structure 45 has, for example, a width W11 (dimension in the width direction X) of 0.2 mm and a height H11 (dimension in the gripping direction Y) protruding from the gripping surfaces 25A and 25B of 0.2 mm (see FIG. 10).

### Configuration of Backing Member

As illustrated in FIG. 6, the backing member 52 includes the base part 61 and the support part 62 described above, and supports the ultrasound oscillator 51 from the outer side (side opposite to the acoustic matching layer 53). The backing member 52 is formed of, for example, a material having rigidity, such as hard rubber or a resin.

The support part 62 is a part that protrudes from the inner surface 61A of the base part 61 and that supports the ultrasound oscillator 51 including the piezoelectric element 54. The support part 62 includes fixed portions 62A and 62B and non-fixed portions 62C and 62D. The fixed portions 62A and 62B and the non-fixed portions 62C and 62D support the ultrasound oscillator 51 at the same position in the gripping direction Y.

The fixed portions 62A and 62B are portions of the support part 62 and are in a state fixed to the ultrasound oscillator 51 including the piezoelectric element 54. The non-fixed portions 62C and 62D are portions of the support part 62 and are in a state not fixed to the ultrasound oscillator 51. The non-fixed portions 62C and 62D are portions different from the fixed portions 62A and 62B and are disposed at positions different from the fixed portions 62A and 62B, and have different shapes from the fixed portions 62A and 62B.

As illustrated in FIG. 11, the fixed portions 62A and 62B are located at the center of the ultrasound oscillator 51 in the longitudinal direction of the ultrasound oscillator 51, that is, the insertion direction Z. A center line CLZ indicates a center line of the ultrasound oscillator 51 in the insertion direction Z. That is, the fixed portions 62A and 62B are disposed at positions overlapping the center line CLZ of the ultrasound oscillator 51 in the insertion direction Z.

Meanwhile, the fixed portions 62A and 62B are disposed at positions equidistant from the center line CLX in the width direction X. The center line CLX indicates a center line of the ultrasound oscillator 51 in the width direction X. That is, the fixed portions 62A and 62B are disposed at positions symmetric to each other with respect to the center line CLX of the ultrasound oscillator 51 in the width direction X (see FIG. 8). As a result, since the ultrasound oscillator 51 is supported and fixed at positions equidistant from the center line CLX, the ultrasound oscillator 51 can vibrate with equal vibration amounts in the width direction X in a case in which the ultrasound oscillator 51 vibrates.

The ultrasound oscillator 51 is fixed to the fixed portions 62A and 62B by, for example, applying an adhesive to positions (portions hatched with oblique lines in FIG. 11) at which the ultrasound oscillator 51 is supported in the fixed portions 62A and 62B and bonding the fixed portions 62A and 62B and the ultrasound oscillator 51. The method of fixing the ultrasound oscillator 51 to the fixed portions 62A and 62B is not limited to this method, and the ultrasound oscillator 51 may be fixed by, for example, screwing or the like. As described above, the fixed portions 62A and 62B are disposed at positions symmetric to each other with respect to the center line CLX in the width direction X. That is, the fixed portions 62A and 62B are fixed to the ultrasound oscillator 51 at positions equidistant from the center line CLX in the width direction X.

The non-fixed portions 62C and 62D are portions different from the fixed portions 62A and 62B in the insertion direction Z, that is, portions other than the center of the ultrasound oscillator 51. Specifically, the non-fixed portions 62C and 62D are divided by a portion including the center line CLZ of the ultrasound oscillator 51 in the insertion direction Z. As described above, the fixed portions 62A and 62B, which are disposed at positions overlapping the center line CLZ of the ultrasound oscillator 51 in the insertion direction Z, are located between the non-fixed portions 62C and 62D.

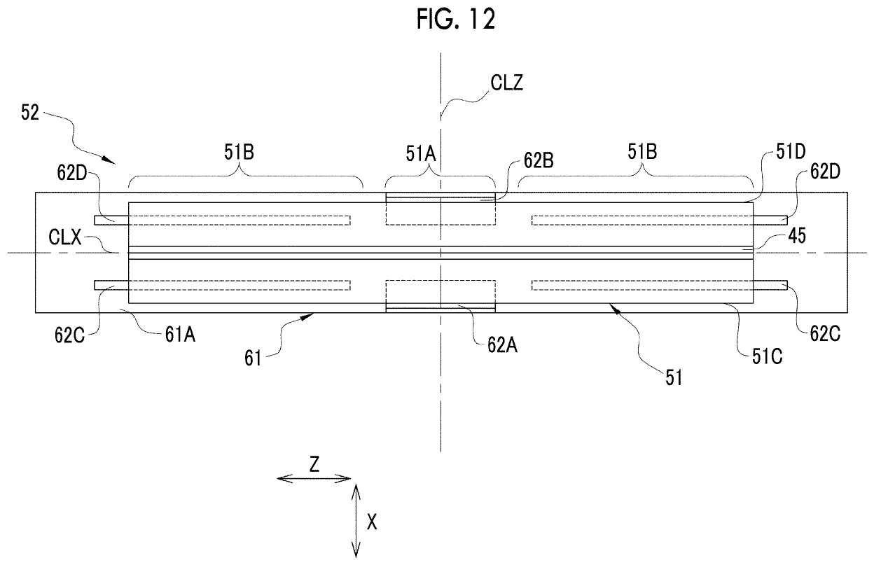

As illustrated in FIG. 12, in a fixed range 51A supported by the fixed portions 62A and 62B in the insertion direction Z, the ultrasound oscillator 51 is fixed and does not vibrate, but in a non-fixed range 51B supported by the non-fixed portions 62C and 62D, the ultrasound oscillator 51 is not fixed and easily vibrates. In particular, in a case in which the surgical treatment apparatus 20 performs the incision treatment, the fixed range 51A of the ultrasound oscillator 51 does not contribute to the incision by the vibration, but the non-fixed range 51B of the ultrasound oscillator 51 is not fixed by the backing member 52 and is not constrained, and thus can vibrate. That is, the non-fixed range 51B can contribute to the incision by the vibration.

The non-fixed portions 62C and 62D support the ultrasound oscillator 51 on the inner side (side on which the center line CLX is located) of end surfaces 51C and 51D of the ultrasound oscillator 51 including the piezoelectric element 54 in the width direction X (see FIG. 7). In this case, a distance DU between the end surfaces 51C and 51D of the ultrasound oscillator 51 and the non-fixed portions 62C and 62D in the width direction X is preferably equal to or greater than 0.3 mm and equal to or less than 1.0 mm, and more preferably equal to or greater than 0.5 mm and equal to or less than 1.0 mm.

In addition, it is preferable that the non-fixed portions 62C and 62D have a hydrophobic surface. As a result, the ultrasound oscillator 51 is less likely to adhere to the non-fixed portions 62C and 62D, and the ultrasound oscillator 51 is likely to vibrate. As the method of making the surfaces of the non-fixed portions 62C and 62D hydrophobic, for example, a paint including a hydrophobic material is applied to the surfaces of the non-fixed portions 62C and 62D. Alternatively, the configuration is not limited to this, and the member including the non-fixed portions 62C and 62D may be formed of, for example, a resin which is a hydrophobic material.

In addition, the base part 61 and the ultrasound oscillator 51 have a constant interval by the support part 62. As a result, an air gap layer 65 (see FIGS. 7 and 8), that is, a gap intervening between the base part 61 and the ultrasound oscillator 51 is formed between the base part 61 and the ultrasound oscillator 51. Since the air gap layer 65 can reflect the ultrasonic waves by the air inside, the air gap layer 65 has a function of reflecting the ultrasonic waves emitted from the back side of the ultrasound oscillator 51. As a result, the ultrasound vibration can be efficiently transmitted to the structure S which is the biological tissue such as the blood vessel.

In a state in which the gripping part 25 is opened, the structure S is passed between the pair of gripping pieces 41A and 41B, and the gripping part 25 is closed to bring the gripping surfaces 25A and 25B close to each other, so that the structure S can be gripped between the pair of gripping pieces 41A and 41B. In a case of performing the treatment such as the sealing, the structure S as a target is gripped by the gripping part 25.

The soft tissue such as the blood vessel contains a large amount of moisture W (see FIGS. 15 to 17), which is a factor of delaying the temperature rise. Therefore, the moisture W contained in the structure S is pressed out by the appropriate gripping (pressurization) of the structure S, and the modification or adhesion of the remaining biological tissue is promoted to improve the sealing effect. An appropriate gripping pressure at which the sealing effect is obtained in the surgical treatment instrument 21 is 10 to 50 N. The moisture W is, for example, a body fluid such as blood.

As illustrated in FIG. 13, the drive device 22 constituting the surgical treatment apparatus 20 comprises a control unit 71, a signal transmitter 72, an amplifier 73, an impedance matching circuit 74, and a frequency monitor 75, and the impedance matching circuit 74 is connected to the ultrasound oscillator 51 of the surgical treatment instrument 21 via the signal cable 76. The control unit 71 controls an amount of power supplied to the piezoelectric element 54. A path connecting the signal transmitter 72, the amplifier 73, the impedance matching circuit 74, and the ultrasound oscillator 51 includes a power supply circuit for driving the piezoelectric element 54. The number of amplifiers 73 and the number of impedance matching circuits 74 may be provided in accordance with the type of the driving frequency to be used.

A program related to various types of processing is stored in a program memory (not illustrated) of the drive device 22. The control unit 71 configured by a processor controls the functions of the signal transmitter 72, the amplifier 73, and the impedance matching circuit 74 by executing the program in the program memory.

The frequency actually used in a case of driving the ultrasound oscillator 51 is displayed on the frequency monitor 75. The signal transmitter 72 has a function of generating the alternating-current voltage signal having any frequency and waveform and has, for example, the same configuration and function as a known function generator. It is preferable that the drive device 22 be provided with a current probe (not illustrated) and a current value monitor (not illustrated) composed of an oscilloscope or the like.

The control unit 71 recognizes the start of the gripping in the surgical treatment instrument 21 by a signal from the gripping detection unit 46, and controls the power supply by two types of driving frequencies in response to the start of the gripping. It is preferable that the control unit 71 also control the gripping in the surgical treatment instrument 21 by operating the actuator 47 while controlling the power supply. The control unit 71 controls the two types of driving frequencies, for example, performs driving at one frequency for a preset time and then automatically switches to another frequency. The control unit 71 also controls whether the gripping in the surgical treatment instrument 21 is maintained or released.

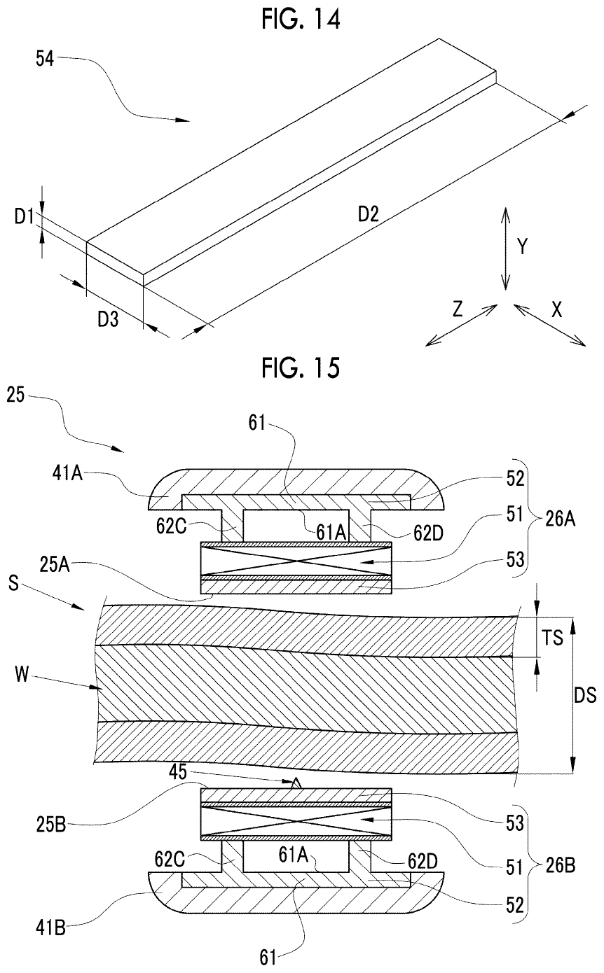

The signal transmitter 72 outputs the alternating-current voltage signal having a predetermined frequency. The predetermined frequency is a frequency at which the ultrasound vibration of the piezoelectric element 54 is maximized, that is, a resonance frequency. The piezoelectric element 54 has the resonance frequency derived from the thickness dimension D1 illustrated in FIG. 14, and, specifically, in a case in which the thickness resonance frequency corresponding to the thickness dimension D1 (m) is denoted by f1 (MHz) and the acoustic velocity (the velocity of the acoustic wave transmitted through the piezoelectric element 54) is denoted by v (m/sec), a relationship represented by D1=v/2f1 is satisfied. It is preferable that the frequency of the thickness resonance frequency f1 be in a range of equal to or higher than 1 MHz and equal to or lower than 10 MHz. The thickness resonance frequency f1 may shift depending on constraint conditions such as a layer configuration of the piezoelectric device or fixation to the backing member, and the thickness resonance frequency in the actual device is a frequency at which the impedance has the local minimum in the vicinity of f1.

In addition, the piezoelectric element 54 has the resonance frequency derived from the length dimension D2 illustrated in FIG. 14, and, specifically, in a case in which the thickness resonance frequency corresponding to the length dimension D2 (m) is denoted by f2 (kHz) and the acoustic velocity is denoted by v (m/sec), a relationship represented by D2=v/2f2 is satisfied. It is preferable that the frequency of the length resonance frequency f2 be in a range of 1 kHz to 1 MHz. The length resonance frequency f2 may be shifted by a constraint condition such as a layer structure of the piezoelectric element 54 or fixation to the backing member 52, and the actual length resonance frequency f2 is a frequency at which the impedance has the local minimum in the vicinity of the above-described range.

The signal transmitter 72 outputs the alternating-current voltage signals of the driving frequencies, for example, the thickness resonance frequency f1 and the length resonance frequency f2 to the amplifier 73. The amplifier 73 amplifies the alternating-current voltage signal output from the signal transmitter 72 to a voltage of a level at which the ultrasound oscillator 51 can be driven. The impedance matching circuit 74 is connected in series to the amplifier 73 and can match the input impedance of the alternating-current voltage signal output from the amplifier 73 with the impedance of the ultrasound oscillator 51.

The current probe measures a current value input from the impedance matching circuit 74 to the ultrasound oscillator 51, and inputs the measured current value to the control unit 71. The control unit 71 displays the current value input from the impedance matching circuit 74 to the ultrasound oscillator 51 on the current value monitor. The control unit 71 controls the signal transmitter 72 so that the ultrasound oscillator 51 is driven by the current value measured by the current probe.

The control unit 71 controls the signal transmitter 72 so that the alternating-current voltage signal for driving the ultrasound oscillator 51 is continuously supplied. In this case, the control unit 71 controls the signal transmitter 72 so that the alternating-current voltage signal is continuously output from the signal transmitter 72 at least while the ultrasound oscillator 51 is driven. The sealing and incision treatments by the surgical treatment instrument 21 are performed by using the driving of the ultrasound oscillator 51.

The impedance matching circuit 74 optimizes the ultrasound vibration efficiency of the ultrasound oscillator 51 at the thickness resonance frequency f1 and the length resonance frequency f2. Meanwhile, the thermal energy is important in addition to the ultrasound energy in the performance of the ultrasound oscillator 51 which is required in the treatments of sealing and incising the structure S such as the blood vessel. Therefore, the ultrasound oscillator 51 is required to implement a driving method for driving the ultrasound oscillator 51 under a condition in which the ultrasound energy and the thermal energy are output, that is, the self-heating of the piezoelectric element 54 is taken into consideration. The control of the amount of heat generated by the piezoelectric element 54 is carried out by adjusting a voltage or a current in a power supply circuit.

The temperature dependence of the thickness resonance frequency f1 and the length resonance frequency f2 of the ultrasound oscillator 51 may become significant due to interference with the environment accompanying the piezoelectric element 54, that is, with the backing member 52, the acoustic matching layer 53, and the like. In such a case, each frequency is set in consideration of a target reach temperature range. Specifically, in a case in which the thickness resonance frequency f1 of the ultrasound oscillator 51 fluctuates in a range from a treatment start temperature to the target reach temperature, the frequency may be set to satisfy an allowable range of the thickness resonance frequency f1 in each temperature range between the treatment start temperature and the target reach temperature of the ultrasound oscillator 51. Further, the same applies to the length resonance frequency f2.

### EXAMPLES

In the present embodiment, an example of performing the sealing and the incision in a case in which the structure S that is the biological tissue is the blood vessel using the surgical treatment apparatus 20 will be described with reference to FIGS. 15 to 17. In the example illustrated in FIGS. 15 to 17, the configuration is the same as the above-described configuration except that the stress concentration structure 45 is provided only on one of the gripping piece 41A or the gripping piece 41B, that is, on the gripping piece 41B. That is, the stress concentration structure 45 is a protrusion that protrudes from the gripping surface 25B, and is not disposed on the gripping surface 25A. In the following description, the same configuration will be used in a case of describing an operation flow of the surgical treatment apparatus 20. The impedance matching circuit 74 uses a first driving frequency and a second driving frequency that are different frequencies. The impedance matching circuit 74 drives the piezoelectric element 54 at the first driving frequency in a case of the sealing and drives the piezoelectric element 54 at the second driving frequency in a case of the incision.

The control unit 71 switches an operation mode among a normal mode before the gripping of the structure S such as the blood vessel, a thickness direction resonance mode in which the impedance matching circuit 74 is driven at the first driving frequency in a case of the pressurized gripping and the sealing, and a length direction resonance mode in which the impedance matching circuit 74 is driven at the second driving frequency in a case of the pressurized gripping and the incision. As the piezoelectric element 54, C-213 (material name; manufactured by FUJI CERAMICS CORPORATION) having the thickness dimension D1 of 0.5 mm, the length dimension D2 of 20 mm, and the width dimension D3 of 3 mm is adopted.

As illustrated in FIG. 15, the structure S that is the biological tissue is the blood vessel, and as an example, a blood vessel having an outer diameter DS of 3 mm and a thickness TS of equal to or greater than 0.5 mm and equal to or less than 0.8 mm is assumed. FIG. 15 illustrates a state in which the gripping part 25 is opened and the structure S is passed between the pair of gripping pieces 41A and 41B, and is a state before the gripping of the structure S.

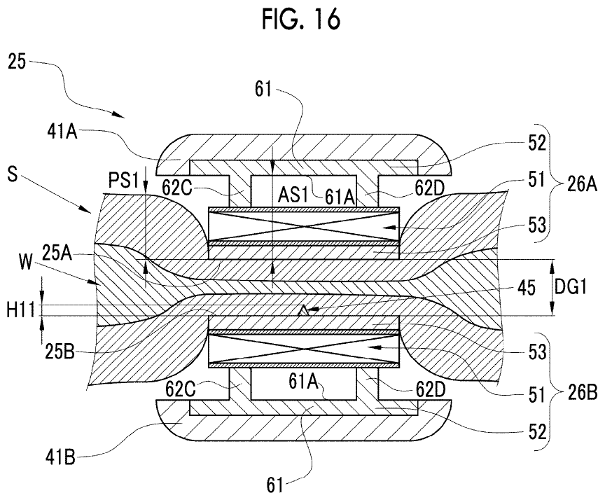

As illustrated in FIG. 16, in a case of starting to grip the structure S using the surgical treatment apparatus 20, a gripping interval DG1 (an interval between the gripping surface 25A and the gripping surface 25B in a case of starting the gripping, and an interval excluding the stress concentration structure 45) is equal to or greater than 1.0 mm and equal to or less than 1.6 mm. In addition, in the example illustrated in FIG. 16, a distance AS1 from the gripping surfaces 25A and 25B to the inner surface 61A of the base part 61 is 1.3 mm, and a protrusion amount PS1 of the structure S from the gripping surface 25A toward the outer side in the gripping direction Y is 1.0 mm, that is, distance AS1>protrusion amount PS1 is assumed.

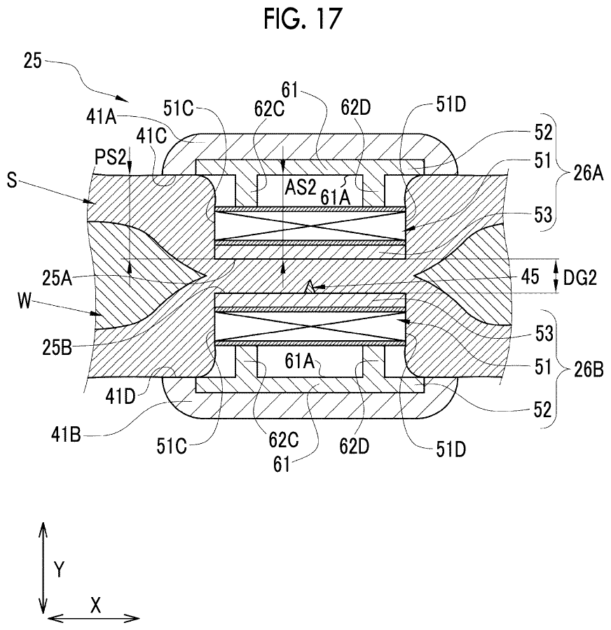

As illustrated in FIG. 17, in a case of performing the sealing of the structure S using the surgical treatment apparatus 20, for example, a gripping interval DG2 (an interval between the gripping surface 25A and the gripping surface 25B in a case of performing the sealing, and an interval excluding the stress concentration structure 45) is equal to or less than 1.0 mm. Further, the height H11 of the stress concentration structure 45 is suppressed in accordance with the gripping interval DG2, and the height H11 is smaller than the gripping interval DG2. In a case of performing the sealing of the structure S, the gripping part 25 is in the closed state.

The non-fixed portions 62C and 62D are disposed at positions at which the structure S does not enter in a case in which the gripping part 25 grips the blood vessel that is the structure S. In the example illustrated in FIG. 17, the distance AS2 from the gripping surfaces 25A and 25B to the inner surface 61A of the base part 61 is 1.3 mm, and the protrusion amount PS2 of the structure S from the gripping surface 25A toward the outer side in the gripping direction Y is 1.5 mm, that is, distance AS2≤protrusion amount PS2 is satisfied. In this case, the structure S is in contact with at least a portion of the inner surface 41C, the inner surface 41D, the inner surface 61A, and the end surfaces 51C and 51D of the ultrasound oscillator 51. On the other hand, in the width direction X, the non-fixed portions 62C and 62D are not located at positions on the outer side of the end surfaces 51C and 51D of the ultrasound oscillator 51. That is, the non-fixed portions 62C and 62D are not located at the positions at which the structure S enters. Further, as described above, the non-fixed portions 62C and 62D support the ultrasound oscillator 51 on the inner side of the end surfaces 51C and 51D of the ultrasound oscillator 51. Therefore, the possibility that the moisture W contained in the structure S enters between the non-fixed portions 62C and 62D and the ultrasound oscillator 51 is very low.

It is preferable that the thickness resonance frequency f1 be mainly used as the first driving frequency, and it is preferable that the length resonance frequency f2 be mainly used as the second driving frequency. The thickness resonance frequency f1 is a resonance frequency derived from the thickness dimension D1 of the piezoelectric element 54, and the length resonance frequency f2 is a resonance frequency derived from the length dimension D2 of the piezoelectric element 54. The impedance matching circuit 74 controlled by the control unit 71 is used for the mode switching. The actual driving frequency need not be strictly the thickness resonance frequency f1 or the length resonance frequency f2.

The control unit 71 detects whether or not the gripping by the gripping part 25 is present and detects the gripping strength (magnitude of a gripping force) from the gripping detection unit 46. The control unit 71 automatically switches and performs the thermal sealing and the driving for incision on the gripped structure S in response to the detection from the gripping detection unit 46. The gripping detection unit 46 can detect the closing operation of changing the gripping part 25 from the opened state to the closed state in the opening/closing operation of the operating part 24 by the user and can detect that the gripping part 25 is gripped with the gripping strength (pressurizing force) of equal to or greater than a certain level.

The control unit 71 automatically switches from the normal mode to the thickness direction resonance mode in response to the detection of the gripping by the gripping detection unit 46, starts the driving at the first driving frequency, and executes the sealing treatment including the sealing of the structure S by the thermal sealing in the first setting period set in advance. The first set period is a period that is required for the sealing treatment of the structure S and that is set in advance by the user, and is, for example, 5 seconds or 10 seconds. During the period of the sealing treatment, the temperature is preferably a temperature after the ultrasound oscillator 51 having the piezoelectric element 54 has reached its maximum temperature. In a case in which the structure S is a biological tissue other than the blood vessel, the treatment is not limited to the sealing treatment, and a cauterization treatment or a coagulation treatment may be performed.

In the thickness direction resonance mode, the structure S is gripped by the gripping part 25, and the self-heating of the piezoelectric element 54 is conducted to the biological tissue together with the start of the predetermined power supply, and the structure S is effectively sealed by the synergistic effects of heat, vibration, and gripping pressure by the application of the ultrasound vibration generated in association therewith. In the thickness direction resonance mode, the power consumption contributing to the self-heating in the piezoelectric element 54 is made larger than the power consumption contributing to the ultrasound vibration, and the impedance adjustment using the impedance matching circuit 74 is used to implement the thickness direction resonance mode.

It is preferable that, in a case of sealing the structure S by the driving at the first driving frequency, the control unit 71 control the actuator 47 to pressurize the structure S more than the time of start of the gripping. During the thermal sealing, the structure S is pressurized to push out the moisture W contained in the structure S at the gripped portion, thereby thinning the structure S. As a result, the incision in the length direction resonance mode can be more effectively performed. The gripping is automatically controlled to be continued or released, but the gripping strength on the structure S can be adjusted in a stepwise manner by the user operation via the operating part 24. It is preferable that the adjustment of the gripping strength in this case be adjustment using a mechanical structure instead of control via a program or the like.

In the thickness direction resonance mode, the control unit 71 optimizes the driving conditions mainly to actively promote the self-heating of the piezoelectric element 54, and drives the piezoelectric element 54 at the thickness resonance frequency f1, and the power consumption contributing to the self-heating is greater than the power consumption contributing to the ultrasound vibration. The power consumption contributing to the self-heating of the piezoelectric element 54 is a difference between the total power consumption and the power consumption contributing to the generation of the ultrasound vibration.

The control unit 71 automatically switches from the thickness direction resonance mode to the length direction resonance mode in response to the end of the first set period, starts driving at the second driving frequency, and executes the incision treatment in a second set period set in advance. The second set period is a period that is required for the incision of the structure S, which has undergone the sealing treatment, and that is set in advance by the user, and is, for example, 3 seconds or 5 seconds. In addition, the gripping of the structure S by the gripping part 25 is controlled by the control unit 71, and the gripping is maintained before and after the switching to the length direction resonance mode. While the gripping is maintained, the gripping strength may be changed, that is, the gripping condition may be changed between the sealing and the incision.

In the length direction resonance mode, the control unit 71 optimizes the driving conditions mainly to actively promote the ultrasound vibration of the piezoelectric element 54, and drives the piezoelectric element 54 at the length resonance frequency f2, and the power consumption contributing to the ultrasound vibration is greater than the power consumption contributing to the self-heating. The ultrasound vibration is transmitted to the structure S, which has undergone a blood vessel sealing treatment, along with stress via the stress concentration structure 45, and the incision treatment is performed. The pressurization may be further performed in the gripping state during the second set period.

The control unit 71 releases the grip and ends the treatment on the structure S in response to the end of the second set period. The control unit 71 switches from the length direction resonance mode to the normal mode by ending the treatment. The surgical treatment apparatus 20 in the normal mode may specify the structure S such as the blood vessel as another treatment target together with the endoscope system 10, and may perform the treatment in the thickness direction resonance mode and the length direction resonance mode in the same manner. In a case of resecting the structure S, the sealing and incision treatments may be performed twice. In this case, one end to be excised is sealed and incised in the first treatment, the other end is sealed and incised in the second treatment, and the target structure S is excised from the subject.

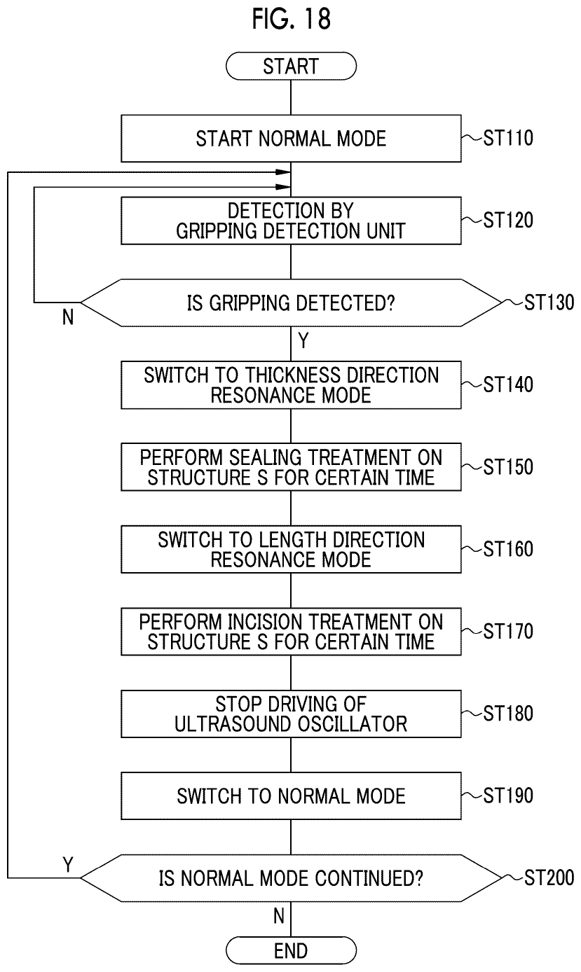

Next, the operation of the surgical treatment apparatus 20 in a case in which the treatment such as the sealing and the incision on the structure S is performed by using the surgical treatment apparatus 20 will be described with reference to the flowchart illustrated in FIG. 18. A power supply of the surgical treatment apparatus 20 is turned on, and the use of the surgical treatment apparatus 20 is started in the normal mode (ST110). In a case in which the normal mode is started, the control unit 71 controls the gripping detection unit 46 to detect the gripping by the gripping detection unit 46 (ST120). The user observes the inside of the subject using the endoscope system 10 combined with the surgical treatment instrument 21, and discovers the structure S to be treated. The user operates the operating part 24 of the surgical treatment instrument 21 to grip the structure S by the gripping part 25. In this case, the control unit 71 detects that the structure S is gripped by the gripping detection unit 46 (ST130: Y). The gripping detection unit 46 repeats the detection until the gripping of the structure S is detected (ST130: N and ST120).

In a case in which the gripping is detected by the gripping detection unit 46, the control unit 71 switches the operation from the normal mode to the thickness direction resonance mode (ST140). In the thickness direction resonance mode, the ultrasound oscillator 51 is driven at the first driving frequency at which the contribution to the self-heating is larger than the contribution to the ultrasound vibration, and the sealing treatment of thermally sealing the structure S is performed for a certain time (ST150). In this case, it is preferable that the control unit 71 perform the sealing treatment by holding a state in which pressurized gripping by the gripping part 25 is maintained by operating the actuator 47 instead of the user operation in the thickness direction resonance mode.

After the thermal sealing for a certain time, the control unit 71 switches from the thickness direction resonance mode to the length direction resonance mode (ST160). In a case in which the switching is performed from the thickness direction resonance mode to the length direction resonance mode, it is preferable that the control unit 71 control the actuator 47 to hold a state in which the gripping by the gripping part 25 is maintained. In the length direction resonance mode, the ultrasound oscillator 51 is driven at the second driving frequency at which the contribution to the ultrasound vibration is larger than the contribution to the self-heating, and the incision treatment of incising the structure S is performed for a certain time (ST170). Since the stress concentration structure 45 on which the stress is concentrated on the structure S vibrates, the incision treatment can be performed on the structure S after the sealing. After the incision treatment for a certain time, the control unit 71 stops the driving of the ultrasound oscillator 51 (ST180).

After stopping the driving of the ultrasound oscillator 51, the control unit 71 switches from the length direction resonance mode to the normal mode (ST190). In a case in which the user wants to continue the treatment, for example, in a case in which there is another structure S to be treated in the subject, the power supply and the control by the drive device 22 are continued. That is, the control unit 71 continues the normal mode (Y in ST200). Then, the process returns to gripping detection by the gripping detection unit 46 (ST120), and in a case in which gripping is detected (Y in ST130), the above-described process is repeated. In a case in which the user wants to end the treatment (N in ST200), the user turns off the power supply of the surgical treatment apparatus 20, and stops the power supply and the control by the drive device 22.

As described above, by switching between the thickness direction resonance mode and the length direction resonance mode to drive the ultrasound oscillator 51 using the surgical treatment apparatus 20, the sealing treatment of the necessary part by the self-heating can be performed on the structure S that is the biological tissue such as the blood vessel, and the sealing part can undergo the incision treatment accurately and easily by the ultrasound vibration after the sealing treatment.

As described above, in the surgical treatment apparatus 20, the backing member 52 is fixed to the ultrasound oscillator 51 in the fixed portions 62A and 62B among the portions that support the ultrasound oscillator 51 including the piezoelectric element 54, and is not fixed to the ultrasound oscillator 51 in the non-fixed portions 62C and 62D. As a result, the portions of the ultrasound oscillator 51 supported by the non-fixed portions 62C and 62D easily vibrate. Therefore, the moisture W contained in the structure S is unlikely to adhere and solidify between the ultrasound oscillator 51 and the non-fixed portions 62C and 62D. That is, even in a case in which the moisture W solidifies by the heating due to the vibration of the ultrasound oscillator 51 in the sealing treatment, the vibration of the ultrasound oscillator 51 during the incision treatment is not hindered. Therefore, the insufficient heating and poor incision of the surgical treatment apparatus 20 can be prevented, and the treatment such as the sealing and the incision can be reliably and accurately performed by the vibration of the ultrasound oscillator 51.

In addition, in a case in which the gripping part 25 grips the blood vessel that is the structure S, the non-fixed portions 62C and 62D of the support part 62 are disposed at positions at which the structure S does not enter. As a result, in a case in which the structure S is gripped, the moisture W contained in the structure S is pushed out, but the moisture W can be prevented from entering between the non-fixed portions 62C and 62D and the ultrasound oscillator 51. Therefore, the moisture W solidified by the heating due to the vibration of the ultrasound oscillator 51 in the sealing treatment can be prevented from hindering the vibration of the ultrasound oscillator 51 during the incision treatment.

Furthermore, in the surgical treatment apparatus 20, the non-fixed portions 62C and 62D support the ultrasound oscillator 51 including the piezoelectric element 54 on the inner side of the end surfaces 51C and 51D of the ultrasound oscillator 51 including the piezoelectric element 54 in the width direction X. As a result, in a case in which the structure S is gripped, the possibility that the moisture W enters between the non-fixed portions 62C and 62D and the ultrasound oscillator 51 is further reduced. Therefore, even in a case in which the moisture W solidifies by the heating due to the vibration of the ultrasound oscillator 51 in the sealing treatment, the vibration of the ultrasound oscillator 51 during the incision treatment is not hindered.

In addition, since the surfaces of the non-fixed portions 62C and 62D are made of a hydrophobic material, the moisture W contained in the structure S is less likely to adhere. Therefore, even in a case in which the moisture W solidifies by the heating due to the vibration of the ultrasound oscillator 51, the vibration of the ultrasound oscillator 51 is not hindered.

In the present embodiment, the ultrasound oscillator 51 is provided on both of the pair of gripping pieces 41A and 41B constituting the gripping part 25, but the present disclosure is not limited to this, and the ultrasound oscillator 51 may be provided on at least one of the pair of gripping pieces 41A and 41B. In this case, the ultrasound oscillator 51 is preferably attached to, for example, the gripping piece 41A, and a member that reflects the ultrasound vibration of the ultrasound oscillator 51 is preferably provided on the gripping piece 41B facing the ultrasound oscillator 51. In addition, the stress concentration structure 45 may be provided on one of the gripping piece 41A or the gripping piece 41B on which the ultrasound oscillator 51 is provided, may be provided on the other of the gripping piece 41A or the gripping piece 41B on which the ultrasound oscillator 51 is not provided, or may be provided on both the gripping pieces 41A and 41B.

In the present embodiment, the hardware structure of the processing units that execute various types of processing, such as the control unit 71, is the following various processors. The various types of processors include a central processing unit (CPU) that is a general-purpose processor functioning as various types of processing units by executing software (program), a graphical processing unit (GPU), a programmable logic device (PLD) that is a processor of which a circuit configuration can be changed after manufacture, such as a field programmable gate array (FPGA), and a dedicated electrical circuit that is a processor of which a circuit configuration is specifically designed to execute various types of processing.

One processing unit may be configured by one of the various processors, or may be configured by a combination of two or more processors of the same type or different types (for example, a plurality of FPGAs, a combination of a CPU and an FPGA, or a combination of a CPU and a GPU). Also, a plurality of the processing units may be configured by one processor. As an example in which the plurality of processing units are configured by one processor, first, there is a form in which one processor is configured by a combination of one or more CPUs and software, and this processor functions as the plurality of processing units, as represented by a computer, such as a client or a server. Second, there is a form in which a processor, which implements the functions of the entire system including the plurality of processing units with one integrated circuit (IC) chip, is used, as represented by a system-on-chip (SoC) or the like. As described above, various processing units are configured by one or more of the various processors described above, as the hardware structure.

Further, the hardware structure of these various processors is, more specifically, an electrical circuit (circuitry) having a form in which circuit elements, such as semiconductor elements, are combined. In addition, the hardware structure of the storage unit is a storage device, such as a hard disc drive (HDD) or a solid-state drive (SSD).

In the above-described embodiment, the endoscope 12 to be combined with the surgical treatment instrument 21 according to the embodiment of the present disclosure is not specified, but any endoscope may be used as long as the endoscope comprises the forceps channel into which the treatment instrument is inserted, and may be, for example, a bronchoscope, an upper gastrointestinal endoscope, or a lower gastrointestinal endoscope.

In the above-described embodiment, the treatment has been described in which the blood vessel is gripped as the biological tissue and sealing and incision are performed, but the biological tissue to be treated is not limited to blood vessels and can also be applied to tubular biological tissues, for example, a portion of the intestine or a portion of the reproductive system.

### EXPLANATION OF REFERENCES

10: endoscope system
12: endoscope
12a: insertion part
12b: operating part
12c: bendable part
12d: distal end part
12e: angle knob
14: light source device
15: processor device
16: display
17: user interface
20: surgical treatment apparatus
21: surgical treatment instrument
22: drive device
23: flexible sheath
24: operating part
25: gripping part
25A, 25B: gripping surface
26A, 26B: ultrasound oscillator unit
31: forceps port
32: forceps channel
33: forceps outlet
41A, 41B: gripping piece
41C, 41D: inner surface
41E, 41F: recess
42: opening/closing mechanism
43: support member
44: operating wire
45: stress concentration structure
45A: side surface
45B: ridgeline
46: gripping detection unit
47: actuator
51: ultrasound oscillator
51A: fixed range
51B: non-fixed range
51C, 51D: end surface
52: backing member
53: acoustic matching layer
54: piezoelectric element
56, 57: electrode layer
61: base part
61A: inner surface
62: support part
62A, 62B: fixed portion
62C, 62D: non-fixed portion
65: air gap layer
71: control unit
72: signal transmitter
73: amplifier
74: impedance matching circuit
75: frequency monitor
76: signal cable
AS1, AS2: distance
CLX: center line
CLZ: center line
D1: thickness dimension
D2: length dimension
D3: width dimension
DG1: gripping interval
DG2: gripping interval
DS: outer diameter
DU: distance
H11: height
PS1, PS2: protrusion amount
S: structure
TS: thickness
W: moisture
W11: width
X: width direction
Y: gripping direction
Z: insertion direction

What is claimed is:

1. A surgical treatment apparatus comprising:
a gripping part that grips a biological tissue;
an ultrasound oscillator that is provided at the gripping part and that includes a piezoelectric element;
a support member that is provided at the gripping part and that supports the piezoelectric element;
an impedance matching circuit for driving the piezoelectric element;
a processor that controls an amount of power supplied to the piezoelectric element; and
a stress concentration structure that is provided at the gripping part and that protrudes from a gripping surface facing the biological tissue,
wherein the impedance matching circuit drives the piezoelectric element using a first driving frequency and a second driving frequency that are different from each other, and
the support member has fixed portions fixed to the piezoelectric element and a non-fixed portion not fixed to the piezoelectric element.

2. The surgical treatment apparatus according to claim 1,
wherein the non-fixed portion is disposed at a position where the biological tissue does not enter when the gripping part grips the biological tissue.

3. The surgical treatment apparatus according to claim 2,
wherein the non-fixed portion supports the piezoelectric element on an inner side of an end surface of the piezoelectric element in a width direction intersecting a vibration direction of the piezoelectric element.

4. The surgical treatment apparatus according to claim 3,
wherein the fixed portions are fixed to the piezoelectric element at positions equidistant from a center line of the piezoelectric element in the width direction.

5. The surgical treatment apparatus according to claim 4,
wherein the gripping part includes a pair of gripping pieces that grip the biological tissue, and
the ultrasound oscillator and the stress concentration structure are provided on at least one of the gripping pieces.

6. The surgical treatment apparatus according to claim 4,
wherein the gripping part includes a pair of gripping pieces that grip the biological tissue, and
the stress concentration structure is provided on at least one of the gripping pieces.

7. The surgical treatment apparatus according to claim 6,
wherein the support member is disposed parallel to a longitudinal direction intersecting the vibration direction of the piezoelectric element and the width direction.

8. The surgical treatment apparatus according to claim 7,
wherein the fixed portions are located at the center of the piezoelectric element in the longitudinal direction.

9. The surgical treatment apparatus according to claim 8,
wherein the non-fixed portion has a hydrophobic surface.

10. The surgical treatment apparatus according to claim 9,
wherein the biological tissue is a blood vessel.
